(19) United States
(12) Patent Application Publication
(10) Pub. No.: US 2025/0041236 A1
(43) Pub. Date: Feb. 6, 2025

(54) METHOD FOR SYNTHESIZING GINSENOSIDE RG3-LOADED NANONIOSOMES

(71) Applicants: Auwal Ibrahim TANKO, Jeddah (SA); Salman HOSAWI, Jeddah (SA); Syed Sarim IMAM, Riyadh (SA); Misbahuddin RAFEEQ, Jeddah (SA); Alaa Hamed HABIB, Jeddah (SA); Muhammad Barkaat HUSSAIN, Jeddah (SA); Mukhtiar BAIG, Jeddah (SA); Imran KAZMI, Jeddah (SA)

(72) Inventors: Auwal Ibrahim TANKO, Jeddah (SA); Salman HOSAWI, Jeddah (SA); Syed Sarim IMAM, Riyadh (SA); Misbahuddin RAFEEQ, Jeddah (SA); Alaa Hamed HABIB, Jeddah (SA); Muhammad Barkaat HUSSAIN, Jeddah (SA); Mukhtiar BAIG, Jeddah (SA); Imran KAZMI, Jeddah (SA)

(21) Appl. No.: 18/923,533

(22) Filed: Oct. 22, 2024

(51) Int. Cl. A61K 9/51; A61K 31/704

(52) U.S. Cl. CPC A61K 9/5123; A61K 9/5192; A61K 31/704

(57) ABSTRACT

The present invention provides a formulation of Ginsenoside Rg3-loaded niosomes enhance its pharmacokinetic properties and improve its anticancer efficacy, both in vitro and in silico, by facilitating targeted delivery and interaction with key cancer-related molecules such as Cathepsin B. The present invention provides a method for synthesizing Ginsenoside Rg3-loaded nanoniosomes, comprising dissolving cholesterol and Span 80 in chloroform to form a lipid phase; dissolving Ginsenoside Rg3 in methanol and transferring the dissolved Ginsenoside Rg3 into the lipid phase; evaporating the chloroform and methanol solvent mixture at a temperature of 40° C. to form a thin lipid film; hydrating the thin lipid film using phosphate-buffered saline (PBS) for 25 minutes at 45° C. to form the nanoniosomal formulation; cooling the formulation overnight; and subjecting the formulation to sonication for 40 minutes to reduce the mean vesicle size.

### FIELD OF THE INVENTION

The present invention provides a formulation of Ginsenoside Rg3-loaded niosomes enhance its pharmacokinetic properties and improve its anticancer efficacy, both in vitro and in silico, by facilitating targeted delivery (due to the desirable properties of niosomes) and interaction with key cancer-related molecules such as Cathepsin B.

### BACKGROUND OF THE INVENTION

Cancer remains an enduring global challenge, with its complex and diverse forms relentlessly affecting millions of lives worldwide. It is regarded as one of the primary causes of mortality globally, contributing to around 10 million fatalities (or roughly one in six deaths) in 2020. Since most current cancer therapies are insufficiently effective to defend against this illness fully, finding the perfect cure remains a challenge. Among the factors contributing to these challenges in treating cancer include the inability to specifically target stem cells, the drug resistance characteristics of tumors, inadequate epigenetic profiling for cancer and low specificity of current epi-drugs, Cancer metastasis, off-target toxicity of conventional cancer therapies, and the lack of reliable biomarkers. While a lot of efforts and progress have been made in this regard, the search for more efficient and less toxic therapeutic approaches continues. Among the several strategies, chemotherapy (alone or in combination with radiotherapy) has stood as a predominant method for cancer treatment. However, its inadequate effectiveness, serious side effects, and the emergence of drug resistance continue to be a challenge.

Therefore, research into alternative therapy approaches has drawn more attention. One of such approach is the possibility of using natural products to treat cancer, which is now a subject of significant research. Among them, ginsenoside Rg3, a phytochemical derived from Panax ginseng, has emerged as a considerable candidate. The root of this Chinese traditional medicine, sometimes known as white or red ginseng, has been widely used in Asian nations for centuries. It shows a range of pharmacological efficacy such as treatment and preventing of disease, and has several pharmacological benefits, including the improvement of memory and learning, tumor growth inhibition, immune function enhancement, and cardiovascular health.

Owing to its content of ginsenoside saponins, ginseng is widely used as medicine. These compounds are responsible for its pharmacological activity. A hydrophobic four-ring steroid backbone joined to sugar moiety at the C20 location is the basic structural component of ginsenosides, and they are found as 20 (R) and 20 (S) epimers, two stereoisomeric forms. Rg3 is unique among the ginsenosides that have undergone extensive research. It has two epimers, 20 (R) ginsenoside Rg3 and 20 (S) ginsenoside Rg3, similar to its counterparts. The transformation of fresh white ginseng into red ginseng (a variant with enhanced medicinal properties) occurs primarily through steam heating for several hours. Higher concentrations of the primary epimer, 20(S)-ginsenoside Rg3, are produced by this procedure. Additionally, enzymatic hydrolysis provides an alternative way of producing 20(S)-ginsenoside Rg3. However, more complex steps are needed to produce 20(R)-ginsenoside Rg3.

Growing evidence suggests that Ginsenoside Rg3 has antiproliferative potential against several forms of cancer, such as breast, liver, and lung cancer. It has been implicated in the proliferation inhibition, apoptosis induction, suppression of angiogenesis, arrest of cell cycle, modulation of metastasis, and enhancement of the immune response against cancer. Furthermore, its versatility extends to acting as an adjuvant to traditional cancer therapies, potentially increasing their effectiveness while reducing side effects through synergistic interactions.

Also, several computational studies such as molecular docking and molecular dynamics simulation studies have shown the diverse potential of ginsenoside Rg3 in cancer treatment by providing important insights into the interactions between Rg3 and key molecular targets involved in cancer development. These studies have shown the ability of Rg3 to bind with high affinity to proteins that play significant roles in tumorigenesis and therapy resistance, such as Glut-1, BIRC5, CA9, SLO1B1, and Cathepsin K.

Though Rg3 has been shown to be effective, there have been reports of inadequate PK characteristics such as solubility issues, inefficient membrane permeability, off-target toxicity, and metabolic instability. By boosting solubility, providing protection, promoting intracellular transport, enabling controlled and sustained release, and evading immune recognition, nanoparticle delivery systems can improve the PK parameters of therapeutic agents, including Ginsenoside Rg3.

Niosomes, widely used as innovative drug delivery vehicles, are vesicles created by mixing non-ionic surfactants—typically from the alkyl or dialkylpolyglycerol ether class—with cholesterol and hydrating them in aqueous medium. As liposomes, niosomes change the organ distribution and metabolic stability of the therapeutic they entrap and extend its circulation. A key difference between the two is that liposomes are made from double-chain phospholipids, which can be charged or neutral, whereas niosomes are made from cholesterol and uncharged single-chain surfactants, and niosomes have a better entrapment effectiveness than liposomes since their cholesterol content is usually higher. Several researchers have used naniosomes to deliver a variety of hydrophilic, hydrophobic drugs, and even nucleic acids to different types of cancer. However, to our knowledge, no research has previously employed nanoniosome to deliver gensenoside Rg3 to cancer cells.

In view of above discussion, therefore there is need to develop and evaluate a Rg3-loaded nanoniosomal formulation for potential anticancer therapy in vitro and using computational approaches.

### SUMMARY OF THE INVENTION

The present invention provides a formulation of Ginsenoside Rg3-loaded niosomes enhance its pharmacokinetic properties and improve its anticancer efficacy, both in vitro and in silico, by facilitating targeted delivery (due to the desirable properties of niosomes) and interaction with key cancer-related molecules such as Cathepsin B.

An objective of the present invention is to develop a Rg3-loaded nanoniosomal formulation using thin film hydration technique.

Another objective of the present invention is to characterize the physical properties of the formulated nanoniosomes, including size, polydispersity index, and morphology.

Another objective of the present invention is to investigate the release profile of Rg3 from the nanoniosomal formulation using the Dialysis method.

Another objective of the present invention is to assess the cytotoxic effects of the Rg3-loaded nanoniosomes on cancer cell lines, particularly breast cancer cell lines, through in vitro assays.

Another objective of the present invention is to conduct molecular docking and molecular dynamics simulations to study the interactions between Rg3 and Cathepsin B, a key enzyme involved in cancer progression.

### BRIEF DESCRIPTION OF FIGURES

These and other features, aspects, and advantages of the present invention will become better understood when the following detailed description is read with reference to the accompanying drawings in which like characters represent like parts throughout the drawings, wherein:

FIG. 1 illustrates Physical characterization of formulations about Size and PDI OF Rg3-loaded Niosome in accordance with an embodiment of the present invention;

FIG. 2 illustrates the in vitro release profile of Ginsenoside Rg3 from nanoiosomes at 37° C., assessed using the dialysis technique in accordance with an embodiment of the present invention;

FIG. 3 represents In vitro Cell viability Assay of T47D breast cancer cells following Rg3 treatment in accordance with an embodiment of the present invention;

FIG. 4 represents MD Simulation and Interaction of Cathepsin B Residues with Rg3. RMSD of protein and ligand over the 50 ns simulation time;

FIG. 5 illustrates Histogram of Protein-Ligand Contacts During the Molecular Dynamics Simulations;

FIG. 6 illustrates a Table about Physical characterization of formulation F2;

FIG. 7 illustrates a Table about Hydrogen bonds formed from the interaction of protein and ligands; and

FIG. 8 illustrates a Table about Hydrophobic interactions formed between Rg3 and Cathepsin B.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present invention. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present invention. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present invention will be described below in detail with reference to the accompanying drawings.

The present invention provides a method for synthesizing Ginsenoside Rg3-loaded nanoniosomes, comprising dissolving cholesterol and Span 80 in chloroform to form a lipid phase; dissolving Ginsenoside Rg3 in methanol and transferring the dissolved Ginsenoside Rg3 into the lipid phase; evaporating the chloroform and methanol solvent mixture using a rotary evaporator at a temperature of 40° C. to form a thin lipid film; hydrating the thin lipid film using phosphate-buffered saline (PBS) for 25 minutes at 45° C. to form the nanoniosomal formulation;
cooling the formulation overnight at room temperature; and subjecting the formulation to sonication for 40 minutes to reduce the mean vesicle size.

In an embodiment, the Ginsenoside Rg3-loaded nanoniosomes are synthesized using a thin-film hydration process.

In another embodiment, the method further comprising determining the size of the nanoniosomes, polydispersity index (PDI), zeta potential (ZP), and entrapment efficiency by dispersing the synthesized nanoniosomes in phosphate buffer; measuring the size, PDI, and ZP using a Zetasizer machine at 25±1° C. with a 90° scattering angle; determining the entrapment efficiency through centrifugation at 15000 rpm for 60 minutes at 4° C., followed by UV spectrophotometric analysis at 345 nm of the supernatant.

In another embodiment, the synthesis process is optimized for enhanced entrapment efficiency of Ginsenoside Rg3 within the nanoniosomes.

In another embodiment, the average size, polydispersity index (PDI), and zeta potential (ZP) of the synthesized Ginsenoside Rg3-loaded nanoniosomes are measured using a Zetasizer machine at 25±1° C. with a 90° scattering angle.

In another embodiment, the synthesized Ginsenoside Rg3-loaded nanoniosomes are dispersed in phosphate-buffered saline (PBS) prior to measuring the vesicle size, PDI, and ZP, and wherein the measurements of the vesicle size, PDI, and ZP are performed in triplicate to ensure consistency in the results.

In another embodiment, the entrapment efficiency of the Ginsenoside Rg3 within the nanoniosomes is determined by centrifuging the synthesized nanoniosomes at 15000 rpm for 60 minutes at 4° C.; separating the supernatant after centrifugation; and determining the Ginsenoside Rg3 content in the supernatant using UV spectrophotometric analysis at 345 nm.

In another embodiment, the supernatant is pipetted out and diluted before conducting the UV spectrophotometric analysis to accurately quantify the Ginsenoside Rg3 content.

In another embodiment, the centrifugation process is configured to optimize separation of the nanoniosomes and unencapsulated Ginsenoside Rg3.

In another embodiment, the cholesterol and Span 80 are dissolved in chloroform in a stepwise manner, first adding Span 80 at 35° C. and maintaining agitation for 10 minutes before the cholesterol is introduced, to ensure uniformity of the lipid phase at a molar ratio of 2.5:1, enhancing lipid bilayer consistency.

In another embodiment, the rotary evaporator is set to a rotational speed between 60-65 rpm, and the evaporation process is conducted at 38° C. under a vacuum of 100 mbar, precisely controlling the evaporation rate to maintain the structural integrity of Ginsenoside Rg3 and prevent premature degradation.

In another embodiment, the phosphate-buffered saline (PBS) used in the hydration process is preconditioned by adjusting its pH to 7.2±0.05 using 0.1 M NaOH and maintaining a temperature of 45° C. to ensure optimal lipid hydration and vesicle formation efficiency.

In another embodiment, the centrifugation speed is incrementally increased in stages, starting at 10,000 rpm for the first 20 minutes, followed by 15,000 rpm for the remaining 40 minutes, to improve separation efficiency and prevent vesicle rupture during the entrapment efficiency measurement process.

In another embodiment, the Zetasizer machine is calibrated using a standard polystyrene nanoparticle solution (200 nm±1 nm) prior to the measurement of vesicle size, PDI, and ZP to ensure accuracy within a margin of error less than 1%, reflecting precise characterization of the synthesized nanoniosomes.

In another embodiment, the molar ratio of cholesterol to Span 80 is adjusted to 2.7:1, based on an iterative vesicle size analysis, to reduce the mean vesicle size to 164.5 nm and achieve a polydispersity index (PDI) of 0.251, optimizing the uniformity and stability of the Ginsenoside Rg3-loaded nanoniosomes.

In another embodiment, the Ginsenoside Rg3-loaded nanoniosomes are subjected to a post-sonication cooling phase under controlled conditions of 20° C. for exactly 12 hours, optimizing vesicle contraction to further reduce vesicle size variability within a range of +3 nm.

In another embodiment, the UV spectrophotometric analysis of the supernatant is carried out after diluting the supernatant with phosphate-buffered saline (PBS) at a ratio of 1:10 to improve the detection sensitivity of Ginsenoside Rg3, with a minimal detection threshold set at 0.01 μg/mL.

In another embodiment, the formulation is sonicated in a pulse-mode configuration, operating at 40 KHz frequency with a 2-second pulse-on and 2-second pulse-off cycle to precisely control the energy input, reducing the mean vesicle size and preventing structural damage to the nanoniosomes.

In another embodiment, the rotary evaporator temperature is precisely maintained at 38.5° C.±0.2° C. using a dual-sensor temperature probe to ensure that the solvent evaporation rate remains constant, preventing localized overheating that could degrade the structural components of Ginsenoside Rg3 during the thin-film formation process.

In another embodiment, the phosphate-buffered saline (PBS) used for hydration is filtered through a 0.22 μm syringe filter immediately before hydration to remove particulate contaminants, ensuring that no extraneous particles interfere with the lipid vesicle formation and contribute to size variability greater than 1.5% during the measurement of the polydispersity index (PDI).

### Materials

Pure Ginsenoside Rg3, lipids, cholesterol, Tween 80, Trypsin, Trypan blue, PBS, FBS, MTT, and dimethylsulfoxide (DMSO) were purchased from Trust Medical Supplier, Jeddah. All the chemicals and reagents used were of standard analytical grade. Human breast cancer cell line, T47D (ATCC, VA, USA), DMEM (BIS BioTech, Saudi), 10% FBS (Sigma, USA) and 1% penicillin (Gibco, USA) were obtained from cryopreserved culture at King Abdulaziz University's Cell Culture Laboratory, Department of Biochemistry, Faculty of Science.

### Methods

### Fabrication of Ginsenoside Rg3-Loaded Nanoniosome

The Ginsenoside Rg3-loaded nanoniosome was prepared using the thin-film hydration process. Cholesterol and Span 80 were dissolved in chloroform to form the lipid phase. Ginsenoside Rg3 was then dissolved in methanol and transferred to the lipid phase. The solvents (chloroform+methanol) were evaporated using a rotary evaporator at 40 degrees Celsius until a thin film formed. The thin film was then hydrated using phosphate-buffered saline (PBS) for 25 minutes at 45 degrees Celsius to obtain the niosomal formulation. The formulation was allowed to cool overnight at room temperature before being subjected to sonication for 40 minutes to reduce the mean size of the vesicles.

### Determination of the Size of Vesicles, PDI, Zeta Potential, and Entrapment Efficiency

The formulation was assessed for its average size, polydispersity index (PDI), and surface charge (zeta potential, ZP). A Zetasizer machine (Malvern Instruments, Worcestershire, UK) was used to measure the vesicles' size, Zetapotential (ZP), and the PDI of the Rg3-loaded nanoniosome formulation at 25±1° C., with a 90° scattering angle. The formulation was dispersed in phosphate buffer for investigation, and the measurements were made in triplicate. Centrifugation of the formulations was carried out at 15000 rpm for 60 minutes at 4° C. to determine the entrapment efficiency of Rg3-loaded nanoniosome formulations. The Rg3 content was determined by UV spectrophotometric analysis at 345 nm after the supernatant was pipetted out and diluted.

### Assessment of In Vitro Release of Rg3

To estimate the Rg3-loaded nanoniosome's in vitro drug release profile, 1 mL of the formulation was placed inside a dialysis bag and then immersed in a PBS solution (maintained at 37° C. and a pH of 7.4) with constant stirring. The PBS solution surrounding the dialysis bag (which contained released Rg3) was extracted at different time intervals for 24 hours, and the absorbance intensity was measured at 203 nm. The quantity of Rg3 measured in the medium is divided by the starting amount of Rg3 in the formulation to determine the amount of Rg3 released.

### Morphological Analysis

For the morphological study of the optimized formulation, scanning electron microscopy (TEM-JOEL JEM1010, Japan) was utilized. Approximately 50 μL of diluted sample was placed onto a copper grid and air-dried for a few minutes. Subsequently, the sample was stained with 1% (w/v) phosphotungstic acid solution and then viewed under a high-resolution electron microscope.

### Cytotoxicity ASSAYS

a. Cell culture: Human breast cancer cell line, T47D (ATCC, VA, USA), which was obtained as a gift from Mahmood Dalhat, was cultured using Dulbecco's modified Eagle media (DMEM) (BIS BioTech, KSA), 10% FBS (Sigma, USA) and 1% penicillin (Gibco, USA). Cells were incubated at 37° C. in 5% CO 2 and the culture media was changed after every 3 days.

b. Cell treatment: 60-80% confluence was achieved after seeding 1×104 cells in 96-well plates for a 24 hr. Next, cells were treated with different serial dilution concentrations of the compound for 24 h.

c. Cell viability assay: Cell viability assay was performed on treated cells using 3-[4,5-dimethylthiazol-2-yl]-2,5 diphenyl tetrazolium bromide (MTT) (Invitrogen, USA). Briefly, 10 μL of MTT (5 mg/mL) was added to each well of the 96-well plate containing treated cells in 90 uL DMEM and incubated for 3 h. Subsequently, the content was removed, and 100 μL of DMSO was added, followed by incubation for 30 min. After incubation, the absorbance of the plate contents was measured using a microplate reader (BioTeK®), and the maximal inhibitory concentration (IC50) was recorded.

d. Cell morphology: Cells of 1×10 4 were seeded in 6 wells plate for 24 h to achieve 50-60% confluence. Next, cells were treated with IC 50 of the compound for 24 h and Nikon light microscope was used to capture the images at ×10 magnification.

e. Statistical analysis: Data are represented as mean±SD. Graphpad Prism 8.0.1 was used for analysis. Dunnett's comparison test was used to compare the groups. A p-value of less than 0.05 was deemed statistically significant.

### Computational Studies

### Preparation of Protein and Ligands.

The structure of Cathepsin B (PDB ID: 1GMY) was sourced from the RCSB PDB database. To prepare it for docking, water molecules were removed, atoms were fixed, protonation was carried out, and protein energy was minimized using the Maestro graphical user interface (Schr€odingerRelease2020-3). The 2D representation of Ginsenoside Rg3 was acquired from the PubChem database in SDF format.

### Molecular Docking and Docking Interaction Analysis

The interaction between protein residues and ligands was investigated using Maestro software. Extra precision (XP) docking was used, with the protein set as rigid and the ligands as flexible. The protein's active site was identified using the Site Map interface in Maestro, guided by published data on RdRp.

### Docking Results Analysis

The interaction between proteins and ligands was analyzed using the Protein-Ligand Interaction Profiler (PLIP) webserver. Hydrogen bond distances were measured using PLIP and Biovia Discovery Studio Visualizer 2020. The binding affinities, visualization, and interactions of ligand-receptor complexes were analyzed using Discovery Studio Visualizer.

### Molecular Dynamic Simulations

The docked Rg3 and cathepsin B structures underwent molecular dynamic simulation (MDS) using Maestro Desmond Module (Schrodinger “Release 2020-3). System Builder tool in Desmond soaked the complex into a 10 Å distance box, filled with TIP4P water model and OPLS3e force field. After neutralizing ions, the complex was equilibrated at 300K, 1.01 bar pressure, and run for 50 ns. The simulation interaction diagram (SID) assessed complex stability using parameters like rGyr, intramolecular HBs, MolSA, SASA, and PSA.

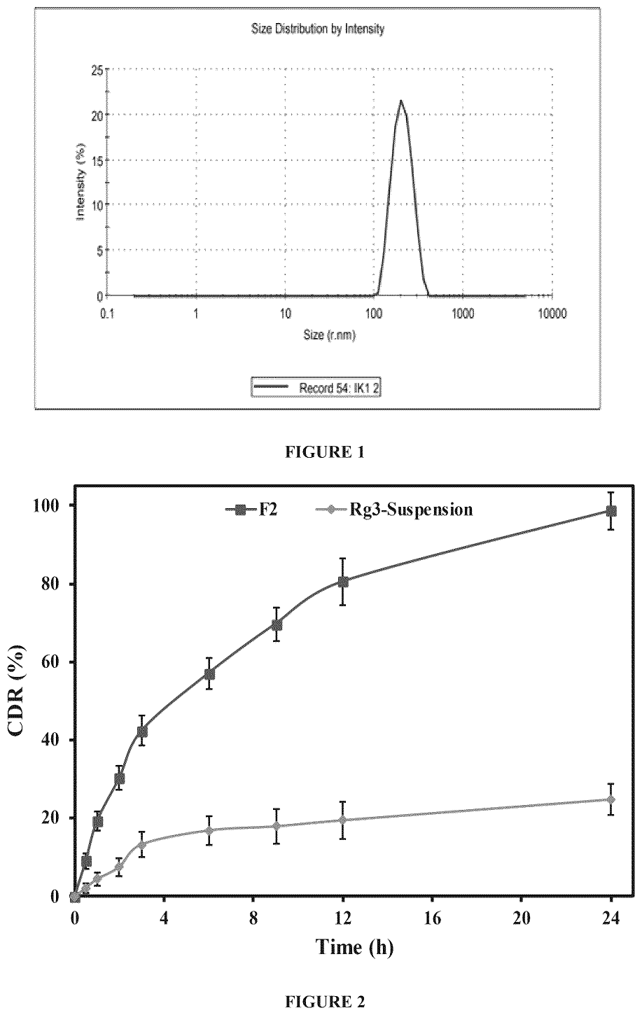

FIG. 1 represents Physical characterization of formulations about Size and PDI OF Rg3-loaded Niosome in accordance with an embodiment of the present invention. Formulations prepared were labeled as F1, F2, and F3. They appeared as round-shaped semi-solids, exhibiting a smooth texture, homogeneity, and a whitish color. Formulation 2, with a pH of 6.9, exhibited the best preparation characteristics (Table in FIG. 6). The shape of the particle is a critical component in determining a micro and nanoparticle formulation's medicinal potential. The surface morphology of the particles was characterized using SEM. We observed uniform and spherical shapes with no change in surface texture, even after the freeze-drying procedure. DLS analysis using a Zetasizer revealed that the chosen formulation (F2) has a particle size of 164.5 nm, a PDI of 0.251, and a zeta potential of −21.2 mV.

FIG. 2 illustrates the in vitro release profile of Ginsenoside Rg3 from nanoiosomes at 37° C., assessed using the dialysis technique. After 24 hours, there was a 98.6% release of the loaded drug (F2), compared to 24.78% for the Free Rg3 suspension. The encapsulation efficiency (EE) of Ginsenoside Rg3 in the niosome formulation was evaluated, resulting in a favorable EE of 86.6%±3.47%.

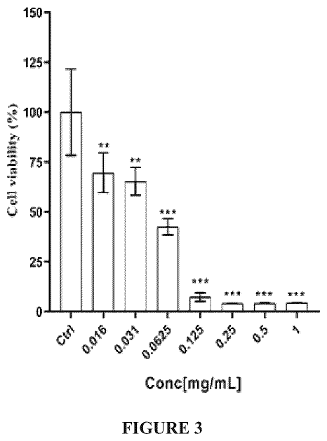

FIG. 3 represents In vitro Cell viability Assay of T47D breast cancer cells following Rg3 treatment in accordance with an embodiment of the present invention. Dose-response experiments were performed to assess the inhibitory effects of Ginsenoside Rg3-loaded nanoniosomes on T47D breast cancer cell lines. It was observed that the cytotoxicity of the Rg3-loaded nanoniosome formulation varied with dose, showing a correlation between increased dose and decreased cell survival rate. The highest concentration tested (1 mg/ml) showed the greatest effect, with only 5% cell viability remaining, while the lowest concentration tested (0.0160 mg/ml) demonstrated the least effect, with 69% cell viability.

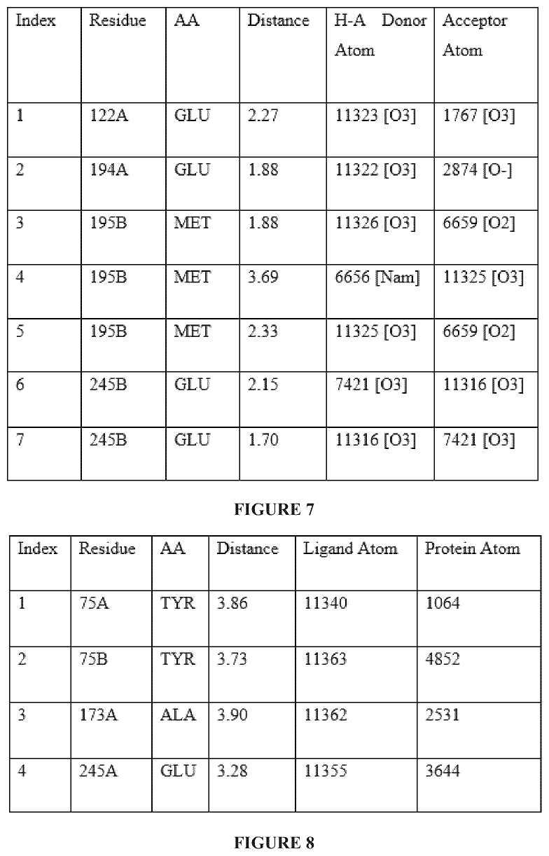

Detailed analysis using the Protein-Ligand Interaction Profiler (PLIP) revealed important interactions essential for stabilization within the binding pocket. Hydrogen bond interactions were observed, with Glutamic Acid residues at positions 122A and 194A forming bonds with Ginsenoside Rg3 oxygen atoms, while the Methionine residue at position 195B engaged in multiple hydrogen bonds with the ligand's oxygen and nitrogen atoms. In addition, Glutamic Acid residue at position 245B contributed significantly to the interactions with Ginsenoside Rg3 as shown in Table in FIG. 7. Furthermore, hydrophobic interactions were detected, with key residues such as Tyrosine at positions 75A and 75B forming contacts with ligand atoms, and Alanine at position 173A and Glutamic Acid at position 245A also participating in hydrophobic interactions with the ligand (Table in FIG. 8).

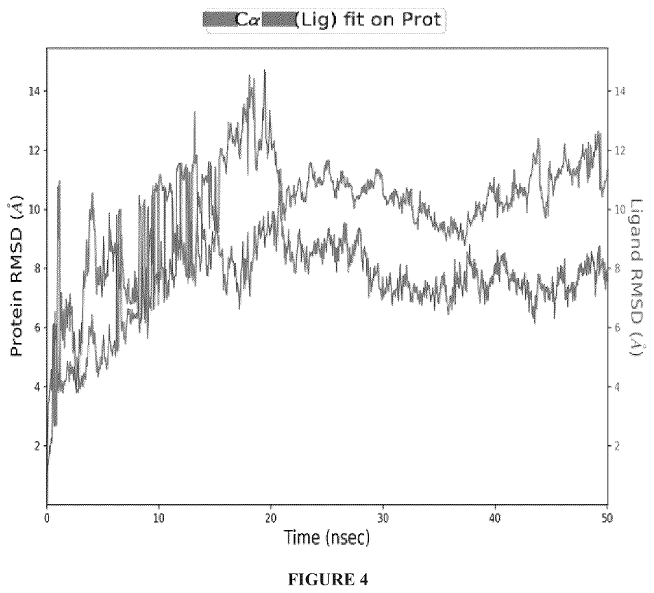

FIG. 4 represents. MD Simulation and Interaction of Cathepsin B Residues with Rg3. RMSD of protein and ligand over the 50 ns simulation time. The molecular dynamics simulation was conducted for the Cathepsin B-Ginsenoside Rg3 complex over a period of 50 nanoseconds (ns). The root mean square deviation (RMSD) plot analysis indicated a flexible interaction between Ginsenoside Rg3 and Cathepsin B, with RMSD values consistently below 15 Å throughout the simulation duration. The protein-ligand complex initially maintained alignment until approximately 16 ns, after which a dissociation occurred, followed by realignment around 20 ns.

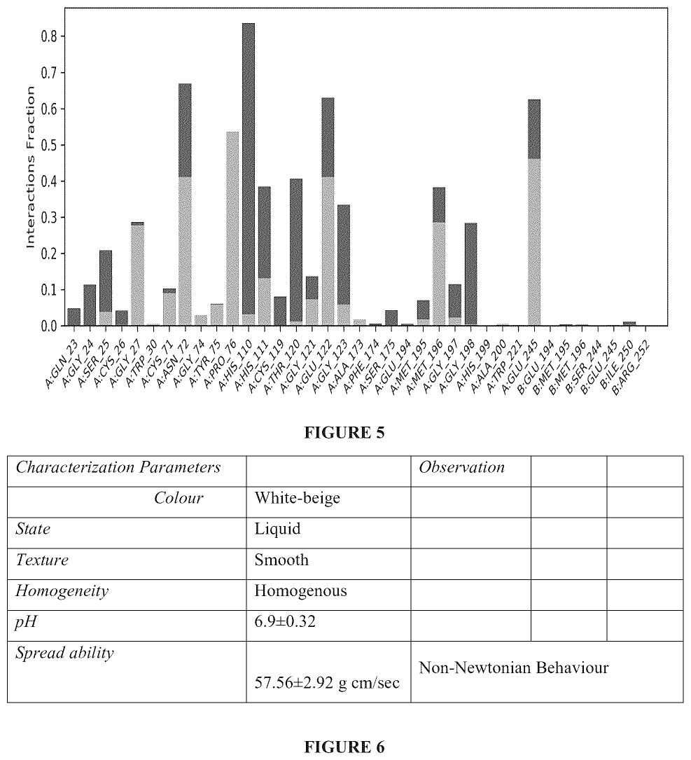

FIG. 5 illustrates Histogram of Protein-Ligand Contacts During the Molecular Dynamics Simulations. Protein-ligand interactions (Hydrogen Bonds, Hydrophobic, Ionic, and Water Bridges) are showned in the normalized stacked bar charts above. A value of 0.7 indicates the interaction is maintained 70% of the simulation time. Values over 1.0 indicate multiple contacts of the same subtype. Subsequent analysis of the protein-ligand contact histogram revealed stable interactions, including a hydrogen bond at chain A residues Asn72, Glu122, and Glu245 persisting for more than half of the simulation time, stable hydrophobic interactions at chain A Pro76 for over 60% of the simulation duration, and a consistent water bridge at chain A His110 for more than 90% of the entire simulation time.

Chemotherapy currently is the most common as well as the primary treatment option for cancer patients. However, its unsatisfactory efficacy, severe side effects, and the emergence of drug resistance continue to be a challenge. Therefore, research into different therapy approaches has drawn more attention. One such approach is the possibility of using natural products to treat cancer, which is now a subject of significant research. Among them, ginsenoside Rg3, a phytochemical derived from Panax ginseng, has emerged as a considerable candidate. Despite Rg3's proven efficacy in vitro, numerous reports have highlighted its poor pharmacokinetic (PK) properties, leading researchers to explore nanotechnology and nanomedicine as a means of addressing this challenge through the use of nano-based delivery systems.

Targeted nano-therapy has shown to be a more successful strategy than traditional anticancer treatments, with less toxicity and improved permeability and retention. It modifies biodistribution and prolongs the plasma half-life of nano-sized medications, which causes a differentiable accumulation of nanoparticles in tumor tissues. When nanoparticles surpass the renal excretion threshold due to their size, they experience an extended plasma half-life as their clearance becomes limited. This phenomenon leads to varying levels of nanoparticle accumulation within tumor tissues, resulting in higher concentrations of the nano-sized drug compared to plasma or other organs. This accumulation is dependent on time and can be consistently observed across tumors of varying sizes.

Niosomes, composed of bilayered vesicles formed by non-ionic surfactants, act as vehicles for various pharmacological substances such as peptides, hormones, antigens, medications, and genetic material. Non-ionic surfactants primarily compose the vesicular layer of niosomes, providing their fundamental structure. Including substances such as cholesterol increases the stiffness of the vesicle's double-layer structure. Pharmacological and immunological responses that cause therapeutic molecules to degrade and become inactive are prevented by this strong carrier system. Resembling cell membranes, results in improved permeation across cellular membranes through osmosis, offering great biocompatibility. Besides, niosomes surpass other drug delivery systems in terms of production cost efficiency and the ability to store chemicals at room temperature.

FIG. 6 illustrates a Table about Physical characterization of formulation F2;

FIG. 7 illustrates a Table about Hydrogen bonds formed from the interaction of protein and ligands; and

FIG. 8 illustrates a Table about Hydrophobic interactions formed between Rg3 and Cathepsin B.

In the present invention, ginsenoside Rg3-loaded nanoniosomes were fabricated using the thin film hydration method with Span 80 and cholesterol as the lipid phase, characterized, and then tested for their anticancer effects on T47D breast cancer cell lines. Several other studies have used different varieties of nanocarriers to improve the PK properties of Rg3 and/or other ginsenosides, alone or in synergism with other therapies. For example, Yu et al. studied liposomes loaded with Rg3 (L-Rg3), revealing stronger cytotoxicity and enhanced tumor inhibition compared to Rg3 solution in vitro and in Wistar rats, with increased pharmacokinetic parameters. Rahimi et al. also synthesized pH-responsive graphene oxide (GO) nanoparticles loaded with DOX and conjugated with Rg3, reducing toxicity while enhancing anticancer efficacy in Huh7 and MDA-MB-231 cell lines through modulation of gene expression. Similarly, Miao et al. developed FPC-Rg3-Liposomes to enhance G-Rg3 solubility and targeting, showing significant 4T1 cell inhibition and reduced tumor development in mice without organ damage. Again, Zare-Zardini and colleagues synthesized Rh2-loaded niosomal formulations and investigated their anticancer activity. Interestingly, the nanoformulation demonstrated improved cytotoxicity and cellular uptake compared to free Rh2.

The results of the present invention suggest that niosomes could effectively deliver Rg3 to cancer cells, showing promise in cancer therapy. The round-shaped semi-solid appearance, smooth texture, homogeneity, and whitish color of the formulations indicate uniformity and stability, which is comparable to the characteristics of Zare-Zardini's niosomal formulation, crucial for reliable drug delivery. Formulation 2, which stands out for having the best preparation qualities and a neutral pH of 6.9, was chosen for further investigation in the present invention. In addition, the particle size of 164.5 nm and polydispersity index (PDI) of 0.251 observed in our formulations highlight their nano-sized nature and uniformity, further supporting their potential as drug delivery vehicles for Rg3. The relatively small particle size and low PDI suggest minimal variation in particle size distribution, which is advantageous for achieving consistent and predictable drug release kinetics and is an indicator of their stability, effectiveness, safety, and in vitro and in vivo behaviour. Moreover, the niosomes' nanoscale size also allows for improved cellular absorption and penetration, which may boost Rg3's bioavailability and therapeutic efficacy against cancer cells. Zeta potential determines the nonaqueous suspension's dispersion control, making it a crucial characteristic for nanoparticles and nanoformulations in addition to particle size distribution. For nanoparticle stability, the ideal zeta potential range is usually +20 to +30 mV or −30 to −20 m. In the present invention, the formulation (F2)'s zeta potential was found to be −21.2 mV in this investigation. This negative value indicates that the particles have a significant surface charge, which is advantageous for preserving a stable suspension and indicating that the formulation is less likely to aggregate over time. This stability is important for consistent drug delivery both in vitro and in vivo and impacts nanoparticle interactions with biological membranes, their biodistribution, and therapeutic efficacy. Our characterization data aligns with Haddadian et al.'s findings, who developed niosomes-loaded selenium nanoparticles for enhanced anticancer, antibacterial, and anti-biofilm activities, with our formulation's particle size slightly more favorable than that of their formulation (177.9 nm).

Despite a lot of research in nanomedicine for cancer treatment over the past decades, only a limited number of nanomedicines have been approved and are available in the market. One contributing factor to this limitation is the challenge in achieving sufficient control over the release pattern of drugs, adequate drug loading, and appropriate release kinetics to deliver therapeutic doses effectively at the intended targeted site. In the present invention, the in vitro release profile of Ginsenoside Rg3 from the nanoniosomes demonstrated sustained and efficient drug release characteristics. The dialysis technique revealed a remarkable 98.6% release of the loaded drug from Formulation 2 after 24 hours, in contrast to the significantly lower release percentage (24.78%) observed for Free Rg3 suspension. This result is consistent with the findings of Xia and colleagues and significantly surpasses the reported release profile in Zare-Zardini and Haddadian's studies. The high release profile recorded can be attributed to the proportion of cholesterol used in our niosome preparation, as Cholesterol content influences the release behavior of niosomes. This enhanced release profile suggests the potential of the nanoniosomal formulations in overcoming the limitations associated with conventional drug delivery systems, such as poor solubility and rapid clearance, thereby improving drug bioavailability and overall therapeutic efficacy. One way to quantify the quantity of a drug that is effectively encapsulated within a carrier nanoparticle delivery system is to look at its encapsulation efficiency (EE). High drug loading or encapsulation efficiency is advantageous as it reduces the need for excipients, allows for higher concentrations of therapeutic agents, decreases the volume of the dose, and shortens the dosing duration. In the present invention, the encapsulation efficiency of Ginsenoside Rg3 in the niosome formulation was found to be 86.6%±3.47%. This high encapsulation efficiency indicates that a large amount of the Ginsenoside Rg3 was successfully loaded into the niosomes, suggesting and maximixing the therapeutic potential of the nanoformulation. Our EE is consistent with the EE efficiency value reported by Xia and colleagues who developed multifunctional ginsenoside Rg3 liposomes and investigated their antitumor potential.

The dose-response experiments on T47D breast cancer cell lines demonstrated the cytotoxic effects of the Ginsenoside Rg3-loaded nanoniosomes. The observed dose-dependent decrease in cell viability, with barely 5% of the cells remaining viable after testing the maximum dose of Rg3-loaded nanoniosomes (1 mg/ml), clearly shows the antitumor potential of the formulation. Not only did the highest dosage show significant cytotoxic effects, but the lowest measured dose (0.0160 mg/ml) also showed nonnegligible effects, with only 69% of the cells viable after 24 hrs of treatment. Although in some respects even better, our cytotoxicity assay data is consistent with that of Yu et al., who developed and evaluated the antitumor activity of Ginsenoside Rg3-loaded liposomes.

An important risk factor for the advancement of cancer and for controlling degenerative processes like apoptosis is the overexpression of a class of lysosomal-encapsulated cellular cysteine proteases, which is represented by cathepsin B. Hence, cysteine cathepsins have become specialised targets in drug design and are employed as useful indicators for cancer and other neurological diseases. To discover the proper drug targeting cathepsin B, many researchers have employed computational approaches, including MD and MD simulations, to assess the interaction of various natural and synthesized compounds with this protein. For example, Saroha and colleagues synthesized eighteen new aurone hybrids decorated with 1,2,3-triazole using click chemistry, achieving excellent yields. They then verified the anticancer potential of these compounds in vitro and proceeded to dock them with cathepsin B using iGEMDOCK software. The docking outcomes showed diverse binding interactions between the ligands and the protein's active sites, including electrostatic, hydrogen bonding, hydrophobic, and van der Waals interactions. In the present invention, the molecular docking study of Cathepsin B with Ginsenoside Rg3 using Maestro software revealed a favorable binding affinity with a docking score of −6.7. Analysis of the docking complex using PLIP showed that hydrogen bond interactions played an important role, with Glutamic Acid residues at positions 122A and 194A forming bonds with Ginsenoside Rg3 oxygen atoms. Furthermore, the Methionine residue at position 195B engaged in multiple hydrogen bonds with the ligand's oxygen and nitrogen atoms. Besides hydrogen bonds, hydrophobic interactions were also observed, with key residues such as Tyrosine at positions 75A and 75B forming contacts with ligand atoms, confirming the potential of Rg3 in targeting this protein and ultimately in cancer therapy. Our molecular dynamics simulation, which lasted for 50 ns showed that Ginsenoside Rg3 and Cathepsin B kept interacting consistently, with RMSD values staying below 15 Å the whole time as proved by the RMSD plot. Initially, the protein-ligand complex stayed together until approximately 16 ns, after which they separated. However, by 20 ns, a realignment occurred, indicating a dynamic interplay between the two molecules. Ginsenoside Rg3's selectivity for certain amino acids within the protein is shown by the evident interaction between it and Cathepsin B, which is depicted throughout the simulation. Next, a thorough examination of these relationships is provided, which shows how stable they are throughout time. Interestingly, Rg3 and several amino acids (Asn72, Glu122, and Glu245) establish a strong hydrogen bond that lasts for a considerable amount of the simulation. Pro76 and His110 exhibit stable hydrophobic contacts and a constant water bridge, respectively, which further contribute to the complex's long-term stability during the simulation. This highlights the potential of Rg3 as a candidate for the therapeutic targeting of Cathepsin B in various diseases including cancer.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible.

1. A method for synthesizing Ginsenoside Rg3-loaded nanoniosomes, comprising:
dissolving cholesterol and Span 80 in chloroform to form a lipid phase;
dissolving Ginsenoside Rg3 in methanol and transferring the dissolved Ginsenoside Rg3 into the lipid phase;
evaporating the chloroform and methanol solvent mixture using a rotary evaporator at a temperature of 40° C. to form a thin lipid film;
hydrating the thin lipid film using phosphate-buffered saline (PBS) for 25 minutes at 45° C. to form the nanoniosomal formulation;
cooling the formulation overnight at room temperature; and
subjecting the formulation to sonication for 40 minutes to reduce the mean vesicle size.

2. The method of claim 1, wherein the Ginsenoside Rg3-loaded nanoniosomes are synthesized using a thin-film hydration process.

3. The method of claim 1, further comprising determining the size of the nanoniosomes, polydispersity index (PDI), zeta potential (ZP), and entrapment efficiency by:
dispersing the synthesized nanoniosomes in phosphate buffer;
measuring the size, PDI, and ZP using a Zetasizer machine at 25±1° C. with a 90° scattering angle;
determining the entrapment efficiency through centrifugation at 15000 rpm for 60 minutes at 4° C., followed by UV spectrophotometric analysis at 345 nm of the supernatant.

4. The method of claim 1, wherein the synthesis process is optimized for enhanced entrapment efficiency of Ginsenoside Rg3 within the nanoniosomes.

5. The method of claim 1, wherein the average size, polydispersity index (PDI), and zeta potential (ZP) of the synthesized Ginsenoside Rg3-loaded nanoniosomes are measured using a Zetasizer machine at 25±1° C. with a 90° scattering angle.

6. The method of claim 6, wherein the synthesized Ginsenoside Rg3-loaded nanoniosomes are dispersed in phosphate-buffered saline (PBS) prior to measuring the vesicle size, PDI, and ZP, and wherein the measurements of the vesicle size, PDI, and ZP are performed in triplicate to ensure consistency in the results.

7. The method of claim 1, wherein the entrapment efficiency of the Ginsenoside Rg3 within the nanoniosomes is determined by:
centrifuging the synthesized nanoniosomes at 15000 rpm for 60 minutes at 4° C.;
separating the supernatant after centrifugation; and
determining the Ginsenoside Rg3 content in the supernatant using UV spectrophotometric analysis at 345 nm.

8. The method of claim 7, wherein the supernatant is pipetted out and diluted before conducting the UV spectrophotometric analysis to accurately quantify the Ginsenoside Rg3 content.

9. The method of claim 7, wherein the centrifugation process is configured to optimize separation of the nanoniosomes and unencapsulated Ginsenoside Rg3.

10. The method of claim 1, wherein the cholesterol and Span 80 are dissolved in chloroform in a stepwise manner, first adding Span 80 at 35° C. and maintaining agitation for 10 minutes before the cholesterol is introduced, to ensure uniformity of the lipid phase at a molar ratio of 2.5:1, enhancing lipid bilayer consistency.

11. The method of claim 1, wherein the rotary evaporator is set to a rotational speed between 60-65 rpm, and the evaporation process is conducted at 38° C. under a vacuum of 100 mbar, precisely controlling the evaporation rate to maintain the structural integrity of Ginsenoside Rg3 and prevent premature degradation.

12. The method of claim 3, wherein the phosphate-buffered saline (PBS) used in the hydration process is preconditioned by adjusting its pH to 7.2±0.05 using 0.1 M NaOH and maintaining a temperature of 45° C. to ensure optimal lipid hydration and vesicle formation efficiency.

13. The method of claim 7, wherein the centrifugation speed is incrementally increased in stages, starting at 10,000 rpm for the first 20 minutes, followed by 15,000 rpm for the remaining 40 minutes, to improve separation efficiency and prevent vesicle rupture during the entrapment efficiency measurement process.

14. The method of claim 5, wherein the Zetasizer machine is calibrated using a standard polystyrene nanoparticle solution (200 nm±1 nm) prior to the measurement of vesicle size, PDI, and ZP to ensure accuracy within a margin of error less than 1%, reflecting precise characterization of the synthesized nanoniosomes.

15. The method of claim 8, wherein the molar ratio of cholesterol to Span 80 is adjusted to 2.7:1, based on an iterative vesicle size analysis, to reduce the mean vesicle size to 164.5 nm and achieve a polydispersity index (PDI) of 0.251, optimizing the uniformity and stability of the Ginsenoside Rg3-loaded nanoniosomes.

16. The method of claim 1, wherein the Ginsenoside Rg3-loaded nanoniosomes are subjected to a post-sonication cooling phase under controlled conditions of 20° C. for exactly 12 hours, optimizing vesicle contraction to further reduce vesicle size variability within a range of ±3 nm.

17. The method of claim 7, wherein the UV spectrophotometric analysis of the supernatant is carried out after diluting the supernatant with phosphate-buffered saline (PBS) at a ratio of 1:10 to improve the detection sensitivity of Ginsenoside Rg3, with a minimal detection threshold set at 0.01 μg/mL.

18. The method of claim 1, wherein the formulation is sonicated in a pulse-mode configuration, operating at 40 kHz frequency with a 2-second pulse-on and 2-second pulse-off cycle to precisely control the energy input, reducing the mean vesicle size and preventing structural damage to the nanoniosomes.

19. The method of claim 9, wherein the rotary evaporator temperature is precisely maintained at 38.5° C.±0.2° C. using a dual-sensor temperature probe to ensure that the solvent evaporation rate remains constant, preventing localized overheating that could degrade the structural components of Ginsenoside Rg3 during the thin-film formation process.

20. The method of claim 3, wherein the phosphate-buffered saline (PBS) used for hydration is filtered through a 0.22 μm syringe filter immediately before hydration to remove particulate contaminants, ensuring that no extraneous particles interfere with the lipid vesicle formation and contribute to size variability greater than 1.5% during the measurement of the polydispersity index (PDI).
